(19) United States
(12) Patent Application Publication
(10) Pub. No.: US 2022/0310231 A1
(43) Pub. Date: Sep. 29, 2022

(54) SYSTEM AND METHOD FOR GENERATING AN ADRENAL DYSREGULATION NOURISHMENT PROGRAM

(71) Applicant: KPN INNOVATIONS, LLC., LAKEWOOD, CO (US)

(72) Inventor: Kenneth Neumann, LAKEWOOD, CO (US)

(73) Assignee: KPN INNOVATIONS, LLC., LAKEWOOD, CO (US)

(21) Appl. No.: 17/216,153

(22) Filed: Mar. 29, 2021

(51) Int. Cl. G16H 20/60; G06N 20/00; G16B 20/20

(52) U.S. Cl. CPC G16H 20/60; G06N 20/00; G16B 20/20

(57) ABSTRACT

A system for generating an adrenal dysregulation nourishment program includes a computing device configured to obtain a biomarker, produce an adrenal enumeration as a function of the biomarker, wherein producing the adrenal enumeration further comprises receiving a homeostatic element, identifying a homeostatic divergence as a function of the biomarker and homeostatic element, and producing the adrenal enumeration as a function of the homeostatic divergence and a statistical deviation, identify an adrenal profile as a function of the adrenal enumeration, wherein producing the adrenal profile further comprises determining an adrenal movement, and producing the adrenal profile as a function of the adrenal enumeration and the adrenal movement using an adrenal machine-learning model, determine an edible as a function of the adrenal profile, and generate a nourishment program as a function of the edible.

### FIELD OF THE INVENTION

The present invention generally relates to the field of artificial intelligence. In particular, the present invention is directed to a system and method for generating an adrenal dysregulation nourishment program.

### BACKGROUND

Current edible suggestion systems do not account for the presence of one or more adrenal functions of an individual. This leads to inefficiency of an edible suggestion system and a poor nutrition plan for the individual. This is further complicated by a lack of uniformity of nutritional plans, which results in dissatisfaction of individuals.

### SUMMARY OF THE DISCLOSURE

In an aspect, a system for generating an adrenal dysregulation nourishment program includes a computing device configured to obtain a biomarker, produce an adrenal enumeration as a function of the biomarker, wherein producing the adrenal enumeration further comprises receiving a homeostatic element, identifying a homeostatic divergence as a function of the biomarker and homeostatic element, and producing the adrenal enumeration as a function of the homeostatic divergence and a statistical deviation, identify an adrenal profile as a function of the adrenal enumeration, wherein producing the adrenal profile further comprises determining an adrenal movement, and producing the adrenal profile as a function of the adrenal enumeration and the adrenal movement using an adrenal machine-learning model, determine an edible as a function of the adrenal profile, and generate a nourishment program as a function of the edible.

In another aspect, a method for generating an adrenal dysregulation nourishment program includes obtaining, by a computing device, a biomarker, producing, by the computing device, an adrenal enumeration as a function of the biomarker, wherein producing the adrenal enumeration further comprises receiving a homeostatic element, identifying a homeostatic divergence as a function of the biomarker and homeostatic element, and producing the adrenal enumeration as a function of the homeostatic divergence and a statistical deviation, identifying, by the computing device, an adrenal profile as a function of the adrenal enumeration, wherein producing the adrenal profile further comprises determining an adrenal movement, and producing the adrenal profile as a function of the adrenal enumeration and the adrenal movement using an adrenal machine-learning model, determining, by the computing device, an edible as a function of the adrenal profile, and generating, by the computing device, a nourishment program as a function of the edible.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary embodiment of a system for generating an adrenal dysregulation nourishment program;

FIG. 2 is a block diagram of an exemplary embodiment of a homeostasis according to an embodiment of the invention;

FIG. 3 is a block diagram of an exemplary embodiment of an edible directory according to an embodiment of the invention;

FIG. 4 is a block diagram of an exemplary embodiment of an adrenal movement according to an embodiment of the invention;

FIG. 5 is a block diagram of an exemplary embodiment of a machine-learning module;

FIG. 6 is a process flow diagram illustrating an exemplary embodiment of a method of generating an adrenal dysregulation nourishment program; and

FIG. 7 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for generating an adrenal dysregulation nourishment program. In an embodiment, this disclosure obtains a biomarker. Aspects of the present disclosure can be used to produce an adrenal enumeration as a function of the biomarker. This is so, at least in part, because the disclosure incorporates a statistical deviation. Aspects of the present disclosure can also be used to identify an adrenal profile using an adrenal machine-learning model. Aspects of the present disclosure can also be used to determine an edible. Aspects of the present disclosure allow for generating a nourishment program. Exemplary embodiments illustrating aspects of the present disclosure are described below in the context of several specific examples.

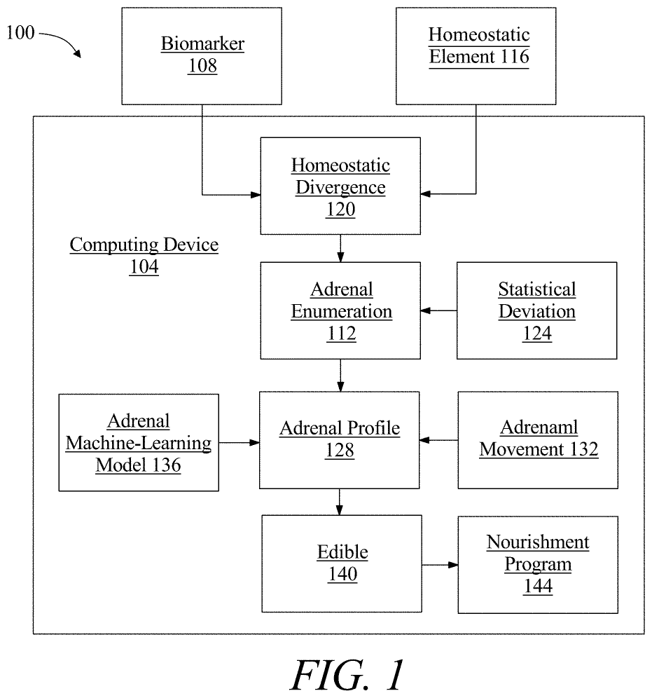

Referring now to FIG. 1, an exemplary embodiment of a system 100 for generating an adrenal dysregulation nourishment program is illustrated. System includes a computing device 104. computing device 104 may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. computing device 104 may include a single computing device operating independently, or may include two or more computing device operating in concert, in parallel, sequentially or the like; two or more computing devices may be included together in a single computing device or in two or more computing devices. computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. computing device 104 may include but is not limited to, for example, a computing device or cluster of computing devices in a first location and a second computing device or cluster of computing devices in a second location. computing device 104 may include one or more computing devices dedicated to data storage, security, distribution of traffic for load balancing, and the like. computing device 104 may distribute one or more computing tasks as described below across a plurality of computing devices of computing device, which may operate in parallel, in series, redundantly, or in any other manner used for distribution of tasks or memory between computing devices. computing device 104 may be implemented using a “shared nothing” architecture in which data is cached at the worker, in an embodiment, this may enable scalability of system 100 and/or computing device.

With continued reference to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, computing device 104 obtains a biomarker 108. As used in this disclosure an “biomarker” is an element of data associated with an individual's adrenal system that denotes a health status, wherein a health status is a measure of the relative level of physical well-being of the adrenal system. In an embodiment, biomarker 108 may denote one or more health status's of an individual's endocrine system. As used in this disclosure an “endocrine system” is a chemical messenger system of an individual's body. For example, and without limitation, endocrine system may include one or more chemical messages associated with growth, development, metabolism, reproduction, and the like thereof. In an embodiment biomarker may include a genetic element. As used in this disclosure a “genetic element” is an element of data associated with the composition of DNA unique to each individual. For example, and without limitation, genetic element may include an element of data denoting the individual has a predisposition for adrenal gland variances as a function of one or more genes such as, but not limited the, ARMC5 gene, GNAS1 gene, CTNNB1 gene, ZNRF3 gene, and the like thereof. In an embodiment, biomarker 108 may include a mutation indicator. As used in this disclosure a “mutation indicator” is an element that denotes a likelihood and/or probability for an individual to have a mutation. For example, and without limitation, mutation indicator may include one or more propensities for mutation due to radiation, electromagnetic waves, chemicals, infectious agents and the like thereof. As a further non-limiting example, mutation indicator may be expressed as a function of a probability such as, but not limited to, a 60% probability that a mutation will occur. As a further non-limiting example, mutation indicator may denote that a gene has a 2% likelihood of mutating. Mutation indicator may include one or more epigenetic elements. As used in this disclosure an “epigenetic element” is an element relating to the change in the health system of an individual as a function of one or more external factors. For example, and without limitation, epigenetic element may include one or more external factors such as traumatic events, illicit drug use, environmental influences, and the like thereof. Mutation indicator may include an inheritance element. As used in this disclosure an “inheritance element” is an element associated with inherited DNA from one or more parents of an individual. For example, and without limitation inheritance element may indicate that a particular region of an individual's DNA was inherited from a mother. As a further non-limiting example, inheritance element may indicate that a particular region of an individual's DNA was inherited from a father. Inheritance element may include one or more lineages, such as a DNA segment from a parent, grandparent, great grandparent, and the like thereof. Inheritance element may be associated with a diploid and/or haploid inheritance of a region of DNA. For example, a first region may be only inherited from a first parent, wherein a second region may be a combination of DNA inherited from the first parent and a second parent.

In an embodiment, and still referring to FIG. 1, biomarker 108 may include a biological sample. As used in this disclosure a “biological sample” is one or more biological specimens collected from an individual. Biological sample may include, without limitation, exhalate, blood, sputum, urine, saliva, feces, semen, and other bodily fluids, as well as tissue. Biomarker 108 may include a biological sampling device. Biomarker 108 may include one or more biological indicators. As used in this disclosure a “biological indicator” is a molecule and/or chemical that identifies the status of an individual's health system. As a non-limiting example, biological indicators may include, Cortisol, ACTH, Cholesterol, Pregnenolone, Progesterone, 11-DOC, Corticosterone, Aldosterone, 11-Deoxycortisol, 170H-Progesterone, 170H-Pregnenolone, DHEA, A4, 11OHA4, 11KA4, Androstenediol, T, 11OHT, 11KT, and the like thereof. As a further non-limiting example, biomarker 108 may include datum from one or more devices that collect, store, and/or calculate one or more lights, voltages, currents, sounds, chemicals, pressures, and the like thereof that are associated with the individual's health status. For example, and without limitation a device may include a/an magnetic resonance imaging device, magnetic resonance spectroscopy device, x-ray spectroscopy device, computerized tomography device, ultrasound device, electroretinogram device, electrocardiogram device, ABER sensor, mass spectrometer, and the like thereof. Additionally or alternatively, biomarker 108 may include any biomarker 108 used as a biomarker as described in U.S. Nonprovisional application Ser. No. 17/136,095, filed on Dec. 29, 2020, and entitled “METHODS AND SYSTEMS FOR DIETARY COMMUNICATIONS USING INTELLIGENT SYSTEMS REGARDING ENDOCRINAL MEASUREMENTS,” the entirety of which is incorporated herein by reference.

Still referring to FIG. 1, computing device 104 may obtain biomarker 108 by receiving an input. As used in this disclosure an “input” is an element of datum that is obtained by an individual relating to the health status of the user. As a non-limiting example input may include a questionnaire and/or survey that identifies a feeling of pain, headache, fever, lethargy, loss of appetite, tenderness, malaise, redness, muscle weakness, and the like thereof. Input may include data from an informed advisor as a function of a medical assessment, wherein a “medical assessment” is an evaluation and/or estimation of the individual's health system. As used in this disclosure “informed advisor” is an individual that is skilled in the health and wellness field. As a non-limiting example an informed advisor may include a medical professional who may assist and/or participate in the medical treatment of an individual's health system including, but not limited to, family physicians, endocrinologists, gastroenterologists, internists, oncologists, pediatricians, cardiologists, geneticists, neurologists, physical therapists, primary care providers, and the like thereof. As a non-limiting example input may include an informed advisor that enters a medical assessment comprising a physical exam, neurologic exam, blood test, urine test, imaging test, cellular and/or chemical analysis, genetic test, measurement, visual examination, and the like thereof. In an embodiment, and without limitation, input may include one or more inputs from a family member. For example, and without limitation, a brother, sister, mother, father, cousin, aunt, uncle, grandparent, child, friend, and the like thereof may enter to computing device 104 that an individual has decreased appetite and/or is exhibiting lethargic tendencies.

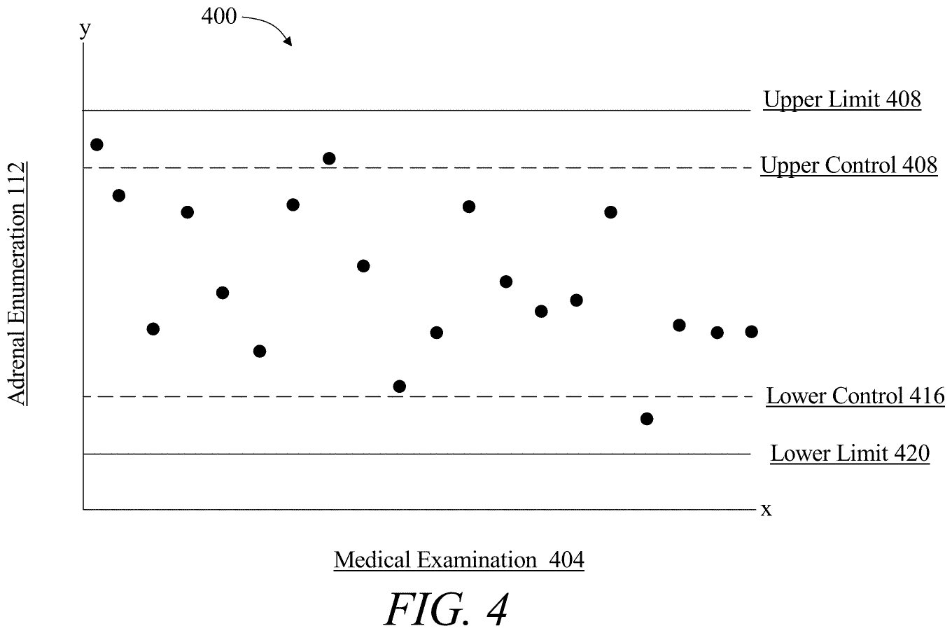

Still referring to FIG. 1, computing device 104 produces an adrenal enumeration 112 as a function of biomarker 108. As used in this disclosure an “adrenal enumeration” is a measurable value associated with the health status of the individual's adrenal glands. As used in this disclosure “adrenal glands” are suprarenal glands that produce a plurality of hormones. For example, and without limitation, adrenal glands may produce one or more hormones to regulate an individual's metabolism, immune system, blood pressure, stress, and the like thereof. As a non-limiting example, adrenal enumeration 112 may be a value of 45 for an adrenal health status of healthy. As a further non-limiting example, adrenal enumeration 112 may be a value of 75 for an adrenal health status of diseased, ill, and/or unwell. Computing device 104 produces adrenal enumeration 112 as a function of receiving a homeostatic element 116. As used in this disclosure a “homeostatic element” is an element of datum representing a mechanism that adjusts an individual's health system to maintain a constant and/or balanced health system, such as but not limited to a homeostatic mechanism and/or a homeostasis. In an embodiment and without limitation, homeostatic element 116 may include a plurality of parts and/or mechanisms such as a receptor, control center, effector, and the like thereof. For example, and without limitation, homeostatic element 116 may include an element of datum denoting the status of homeostasis. For example, and without limitation, homeostatic element 116 may include datum denoting the status of homeostasis such as, but not limited to, maintaining equilibrium of an individual's body temperature, fluid balance, pH range, and the like thereof. As a further non-limiting example, homeostatic element 116 may include datum denoting the status of homeostasis such as, but not limited to, regulation and/or control of core temperatures, blood glucose, iron levels, copper regulation, blood gas levels, blood oxygen content, calcium levels, sodium concentrations, potassium concentrations, fluid balances, energy balance, and the like thereof. As a further non-limiting example, homeostatic element 116 may include datum associated with a renin-angiotensin mechanism. As used in this disclosure a “renin-angiotensin mechanism” is a hormone mechanism that regulates blood pressure, fluid, electrolyte balance, and/or systemic vascular resistance. For example, and without limitation, renin-angiotensin mechanism may include one or more mechanisms associated with sympathetic activity, tubular sodium, water retention, aldosterone secretion, vasoconstriction, ADH secretion, angiotensinogen secretion, renin secretion, angiotensin I secretion, ACE secretion, angiotensin II secretion, and the like thereof. Additionally or alternatively, homeostasis may include a positive feedback loop. As used in this disclosure a “positive feedback loop” is a process where an output of a reaction leads to an increase of the reaction. For example, and without limitation, positive feedback loop may include the production of oxytocin. In an embodiment homeostasis may include a negative feedback loop, wherein a negative feedback loop is a feedback system wherein an output signals to the process and/or mechanism to stop producing the output, as described in detail below, in reference to FIG. 4.

Still referring to FIG. 1, computing device 104 identifies a homeostatic divergence 120 as a function of biomarker 108 and homeostatic element 116. As used in this disclosure a “homeostatic divergence” is a quantitative value comprising the magnitude of divergence of a biomarker from homeostasis. As a non-limiting example, homeostatic divergence 120 may be 2.7 for a homeostasis that may denote that 520-200 mcg/day of aldosterone should be produced, wherein biomarker 108 identifies that an individual has produced 10 mcg/day for the last month. As a further non-limiting example, homeostatic divergence 120 may be 1.2 for a homeostasis that may denote that 5.7-7.4 mg/m²/day of cortisol should be produced, wherein biomarker 108 identifies that an individual has produced 9.6 mg/m²/day for the last 2 weeks. Homeostatic divergence 120 may be determined as a function of receiving a divergence threshold. As used in this disclosure a “divergence threshold” is a parameter that identifies one or more variance limits of the biomarker from homeostasis. In an embodiment, homeostatic divergence 120 may be determined as a function of biomarker 108, homeostasis, and divergence threshold. As a non-limiting example, divergence threshold may determine that an individual is imbalanced with respect to a homeostasis for when the biomarker for epinephrine exceeds 0.5 mg. Computing device 104 produces adrenal enumeration 112 as a function of homeostatic divergence 120 and a statistical deviation 124. As used in this disclosure a “statistical deviation” is a measure of difference between the observed homeostatic divergence and a statistical value computed from a plurality of values in a sample and/or population. For example, and without limitation, a statistical value may be a calculated mean, mode, median, probability distribution, and the like thereof of a plurality of previous biomarkers and/or medical examinations from an individual. As a further non-limiting example, a statistical value may be a calculated mean, mode, median, probability distribution, and the like thereof of a plurality of previous biomarkers and/or medical examinations from a group of individuals. For example, and without limitation, a group of individuals may include one or more groups denoted by demographics, such as but not limited to, race, age, ethnicity, gender, marital status, income, education, employment, and the like thereof. In an embodiment, and without limitation, statistical deviation 124 may include an unsigned deviation, mean signed deviation, dispersion, normalization, standard deviation, average absolute deviation, median absolute deviation, maximum absolute deviation, and the like thereof. As a non-limiting example, homeostatic divergence 120 may be one standard deviation from a statistical value representing a mean blood pressure of individuals ranging from 30-40 years old.

In an embodiment, and still referring to FIG. 1, computing device 104 may produce adrenal enumeration 112 as a function of determining an origin of malfunction. As used in this disclosure an “origin of malfunction” is one or more origination location within the individual's body that caused the imbalance of the homeostasis. For example, and without limitation, an origin of malfunction may denote that an androgenic steroid over production of the adrenal glands originated from a pituitary overstimulation and not from a malfunction at the adrenal glands. As a further non-limiting example, origin of malfunction may denote that a vasoconstriction of arterioles in the extremities originated from an overproduction of cortisol in the adrenal glands and not from a malfunction in another location of the homeostatic element. Computing device 104 may produce adrenal enumeration 112 as a function of biomarker 108 and origin of malfunction using an origin machine-learning model. As used in this disclosure an “origin machine-learning model” is a machine-learning model to produce an adrenal enumeration output given biomarkers and origins of malfunction as inputs; this is in contrast to a non-machine learning software program where the commands to be executed are determined in advance by a user and written in a programming language. Origin machine-learning model 1 may include one or more origin machine-learning processes such as supervised, unsupervised, or reinforcement machine-learning processes that computing device 104 and/or a remote device may or may not use in the determination of adrenal enumeration 112. As used in this disclosure “remote device” is an external device to computing device 104. An origin machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train origin machine-learning process as a function of an origin training set. As used in this disclosure “origin training set” is a training set that correlates a biomarker and/or origin of malfunction to an adrenal enumeration. For example, and without limitation, a biomarker of pregnenolone and an origin of malfunction of the hypothalamus may relate to an adrenal enumeration of 2. As a further non-limiting example, a biomarker of cortisol and an origin of malfunction of the adrenal glands may relate to an adrenal enumeration of 91. Origin training set may be received as a function of user-entered valuations of biomarkers, origins of malfunction, and/or adrenal enumerations. Computing device 104 may receive origin training set by receiving correlations of biomarkers, and/or origins of malfunction that were previously received and/or determined during a previous iteration of determining adrenal enumerations. Origin training set may be received by one or more remote devices that at least correlate a biomarker and/or origin of malfunction to an adrenal enumeration, wherein a remote device is an external device to computing device 104, as described above. Origin training set may be received in the form of one or more user-entered correlations of a biomarker and/or origin of malfunction to an adrenal enumeration. A user may include an informed advisor, wherein an informed advisor may include, without limitation, family physicians, endocrinologists, gastroenterologists, internists, oncologists, pediatricians, cardiologists, geneticists, neurologists, physical therapists, primary care providers, and the like thereof.

Still referring to FIG. 1, computing device 104 may receive origin machine-learning model from a remote device that utilizes one or more origin machine learning processes, wherein a remote device is described above in detail. For example, and without limitation, a remote device may include a computing device, external device, processor, and the like thereof. Remote device may perform the origin machine-learning process using the origin training set to generate adrenal enumeration 112 and transmit the output to computing device 104. Remote device may transmit a signal, bit, datum, or parameter to computing device 104 that at least relates to adrenal enumeration 112. Additionally or alternatively, the remote device may provide an updated machine-learning model. For example, and without limitation, an updated machine-learning model may be comprised of a firmware update, a software update, an origin machine-learning process correction, and the like thereof. As a non-limiting example a software update may incorporate a new biomarker that relates to a modified origin of malfunction. Additionally or alternatively, the updated machine learning model may be transmitted to the remote device, wherein the remote device may replace the origin machine-learning model with the updated machine-learning model and determine the adrenal enumeration as a function of the biomarker using the updated machine-learning model. The updated machine-learning model may be transmitted by the remote device and received by computing device 104 as a software update, firmware update, or corrected origin machine-learning model. For example, and without limitation origin machine-learning model may utilize a random forest machine-learning process, wherein the updated machine-learning model may incorporate a gradient boosting machine-learning process. Updated machine learning model may additionally or alternatively include any machine-learning model used as an updated machine learning model as described in U.S. Nonprovisional application Ser. No. 17/106,658, filed on Nov. 30, 2020, and entitled “A SYSTEM AND METHOD FOR GENERATING A DYNAMIC WEIGHTED COMBINATION,” the entirety of which is incorporated herein by reference.

In an embodiment and without limitation, origin machine-learning model may include a classifier. A “classifier,” as used in this disclosure is a machine-learning model, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Computing device 104 and/or another device may generate a classifier using a classification algorithm, defined as a processes whereby a computing device 104 derives a classifier from training data. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

Still referring to FIG. 1, computing device 104 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(AB) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 104 may then calculate a likelihood table by calculating probabilities of different data entries and classification labels. Computing device 104 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 1, computing device 104 may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 1, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least one value. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute/as derived using a Pythagorean norm: l=\sqrt{Σᵢ₌₀ⁿ aᵢ²}, where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

Still referring to FIG. 1, computing device 104 identifies an adrenal profile 128 as a function of adrenal enumeration 112. As used in this disclosure an “adrenal profile” is a profile and/or estimation of an individual's adrenal gland health status. For example, and without limitation, adrenal profile 128 may denote that an individual's adrenal function is outputting lower than normal estrogen quantities. As a further non-limiting example, adrenal profile 128 may denote that an individual's adrenal function is not binding to and/or receiving signals from an adrenocorticotropic hormone signal. Adrenal profile 128 is identified as a function of determining an adrenal movement 132. As used in this disclosure an “adrenal movement” is a trend and/or movement of an individual's adrenal functions, as described below in further detail, in reference to FIG. 4. For example, and without limitation, adrenal movement may include a positive trend, which may denote improved adrenal gland functioning, a negative trend, which may denote worsening and/or impaired adrenal gland functioning, and/or a neutral trend, which may denote no change and/or alteration to the previous functioning of the adrenal gland.

Still referring to FIG. 1, computing device 104 identifies adrenal profile 128 as a function of adrenal enumeration 112 and adrenal movement 132 using an adrenal machine-learning model 136. As used in this disclosure, an “adrenal machine-learning model” is a machine-learning model to produce an adrenal profile output given adrenal enumeration 112 and/or adrenal movement 132 as inputs; this is in contrast to a non-machine learning software program where the commands to be executed are determined in advance by a user and written in a programming language. Adrenal machine-learning model 136 may include one or more adrenal machine-learning processes such as supervised, unsupervised, or reinforcement machine-learning processes that computing device 104 and/or a remote device may or may not use in the determination of adrenal profile 128. An adrenal machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train adrenal machine-learning process as a function of an adrenal training set. As used in this disclosure, a “adrenal training set” is a training set that correlates at least an adrenal movement and an adrenal enumeration to an adrenal profile. As a non-limiting example an adrenal movement of a negative trend of adrenal functioning may be relate to an adrenal enumeration of 23 for cortisol production wherein an adrenal profile of decreasing and/or reduced adrenal functioning may be outputted. The adrenal training set may be received as a function of user-entered valuations of adrenal movements, adrenal enumerations, and/or adrenal profiles. Computing device 104 may receive adrenal training set by receiving correlations of adrenal movements and/or adrenal enumerations that were previously received and/or determined during a previous iteration of determining adrenal profiles. The adrenal training set may be received by one or more remote devices that at least correlate an adrenal movement and/or adrenal enumeration to an adrenal profile, wherein a remote device is an external device to computing device 104, as described above. The adrenal training set may be received in the form of one or more user-entered correlations of an adrenal movement and adrenal enumeration to an adrenal profile. Additionally or alternatively, a user may include an informed advisor, wherein an informed advisor may include, without limitation, family physicians, endocrinologists, gastroenterologists, internists, oncologists, pediatricians, cardiologists, geneticists, neurologists, physical therapists, primary care providers, and the like thereof.

Still referring to FIG. 1, computing device 104 may receive adrenal machine-learning model 136 from the remote device that utilizes one or more adrenal machine learning processes, wherein a remote device is described above in detail. For example, and without limitation, a remote device may include a computing device, external device, processor, and the like thereof. The remote device may perform the adrenal machine-learning process using the adrenal training set to generate adrenal profile and transmit the output to computing device 104. The remote device may transmit a signal, bit, datum, or parameter to computing device 104 that at least relates to adrenal profiles. Additionally or alternatively, the remote device may provide an updated machine-learning model. For example, and without limitation, an updated machine-learning model may be comprised of a firmware update, a software update, an adrenal machine-learning process correction, and the like thereof. As a non-limiting example a software update may incorporate a new adrenal movement that relates to a modified adrenal enumeration. Additionally or alternatively, the updated machine learning model may be transmitted to the remote device, wherein the remote device may replace the adrenal machine-learning model with the updated machine-learning model and determine the adrenal profile as a function of the adrenal movement using the updated machine-learning model. The updated machine-learning model may be transmitted by the remote device and received by computing device 104 as a software update, firmware update, or corrected adrenal machine-learning model. For example, and without limitation adrenal machine-learning model may utilize a Naïve bayes machine-learning process, wherein the updated machine-learning model may incorporate decision tree machine-learning process. Additionally or alternatively, adrenal machine-learning model 136 may determine the adrenal profile as a function of one or more classifiers, wherein a classifier is described above in detail.

Still referring to FIG. 1, computing device 104 may identify adrenal profile as a function of determining a physiological alteration. As used in this disclosure a “physiological alteration” is an alteration and/or impact the adrenal gland has on the health status of an individual. For example, and without limitation, physiological alteration may include an alteration on the heart as a function of the adrenal gland overproduction and/or secretion of cortisol, wherein cortisol may increase the blood pressure of an individual's circulatory system. As a further non-limiting example, physiological alteration may include an alteration in the musculoskeletal system as a function of reduced production and/or synthesis of androgen by the adrenal glands. In an embodiment, computing device 104 may determine physiological alteration as a function of receiving a target function. As used in this disclosure a “target function” is a recommendation and/or guideline for an individual's biological system. As a non-limiting example, target function may include a recommendation that a blood pressure should be 120/80 mmHg. As a further non-limiting example target function may include a recommendation that a respiratory rate should be 14 breaths per minute. Target function may include recommendations from one or more peer review sources such as scholarly peer reviews, government peer reviews, medical peer reviews, technical peer reviews, and the like thereof. Peer review sources may include, but are not limited to, Nature, The New England Journal of Medicine, The American Journal of Medicine, Journal of American Medical Association, The Lancet, and the like thereof. Target function may include recommendations from one or more informed advisor associations, such as a source of one or more committees, organizations, and/or groups capable of determining and/or organizing recommendations and/or guidelines. As a non-limiting example informed advisor associations may include the American Medical Association, American Nurses Association, The Association for Accessible Medicines and the like thereof. Target function may include recommendations from one or more medical websites that establish guidelines for the health status of individuals. As a non-limiting example, medical website may include, but are not limited to, UMDF, Medline Plus, Mayo Clinic, WebMD, Health.gov, and the like thereof.

Still referring to FIG. 1, computing device 104 may determine physiological alteration as a function of target function and adrenal enumeration 112 using a physiological machine-learning model. As used in this disclosure a “physiological machine-learning model” is a machine-learning model to produce a physiological alteration output given target functions and/or adrenal enumerations as inputs; this is in contrast to a non-machine learning software program where the commands to be executed are determined in advance by a user and written in a programming language. Physiological machine-learning model may include one or more physiological machine-learning processes such as supervised, unsupervised, or reinforcement machine-learning processes that computing device 104 and/or a remote device may or may not use in the determination of physiological alteration, wherein a remote device is an external device to computing device 104 as described above in detail. A physiological machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train physiological machine-learning process as a function of a physiological training set. As used in this disclosure a “physiological training set” is a training set that correlates at least a target function and an adrenal enumeration to a physiological alteration. For example, and without limitation, target function of a blood glucose level of 140 mg/dL and an adrenal enumeration 47 for epinephrine production may relate to a physiological alteration of enhanced blood glucose concentrations. The physiological training set may be received as a function of user-entered valuations of target functions, adrenal enumerations, and/or physiological alterations. Computing device 104 may receive physiological training set by receiving correlations of target functions and/or adrenal enumerations that were previously received and/or determined during a previous iteration of determining physiological alterations. The physiological training set may be received by one or more remote devices that at least correlate a target function and adrenal enumeration to a physiological alteration, wherein a remote device is an external device to computing device 104, as described above. Physiological training set may be received in the form of one or more user-entered correlations of a target function and/or adrenal enumeration to a physiological alteration. Additionally or alternatively, a user may include an informed advisor, wherein an informed advisor may include, without limitation, family physicians, endocrinologists, gastroenterologists, internists, oncologists, pediatricians, cardiologists, geneticists, neurologists, physical therapists, primary care providers, and the like thereof.

Still referring to FIG. 1, computing device 104 may receive physiological machine-learning model from a remote device that utilizes one or more physiological machine learning processes, wherein remote device is described above in detail. For example, and without limitation, remote device may include a computing device, external device, processor, and the like thereof. Remote device may perform the physiological machine-learning process using the physiological training set to generate physiological alteration and transmit the output to computing device 104. Remote device may transmit a signal, bit, datum, or parameter to computing device 104 that at least relates to physiological alteration. Additionally or alternatively, the remote device may provide an updated machine-learning model. For example, and without limitation, an updated machine-learning model may be comprised of a firmware update, a software update, a physiological machine-learning process correction, and the like thereof. As a non-limiting example a software update may incorporate a new target function that relates to a modified adrenal enumeration. Additionally or alternatively, the updated machine learning model may be transmitted to the remote device, wherein the remote device may replace the physiological machine-learning model with the updated machine-learning model and determine the physiological as a function of the adrenal enumeration using the updated machine-learning model. The updated machine-learning model may be transmitted by the remote device and received by computing device 104 as a software update, firmware update, or corrected physiological machine-learning model. For example, and without limitation a physiological machine-learning model may utilize a neural net machine-learning process, wherein the updated machine-learning model may incorporate polynomial regression machine-learning process. Updated machine learning model may additionally or alternatively include any machine-learning model used as an updated machine learning model as described in U.S. Nonprovisional application Ser. No. 17/106,658, filed on Nov. 30, 2020, and entitled “A SYSTEM AND METHOD FOR GENERATING A DYNAMIC WEIGHTED COMBINATION,” the entirety of which is incorporated herein by reference. In an embodiment, and without limitation, physiological machine-learning model may identify physiological alteration as a function of one or more classifiers, wherein a classifier is described above in detail.

In an embodiment, and still referring to FIG. 1, computing device 104 may identify adrenal profile 128 as a function of determining a probabilistic vector. As used in this disclosure a “probabilistic vector” is a data structure that represents one or more a quantitative values and/or measures of probability associated with developing adrenal gland modifications. For example, and without limitation, probabilistic vector may indicate that an individual's adrenal gland function has a high probability of declining rapidly. As a further non-limiting example, probabilistic vector may indicate that an individual's adrenal gland function has a high likelihood of severely altering the health status of the individual. In an embodiment, and without limitation, a vector may be represented as an n-tuple of values, where n is one or more values, as described in further detail below; a vector may alternatively or additionally be represented as an element of a vector space, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm: l=\sqrt{Σᵢ₌₀ⁿ aᵢ²}, where aᵢ is attribute number i of the vector. Scaling and/or normalization may function to make vector comparison independent of absolute quantities of attributes, while preserving any dependency on similarity of attributes.

In an embodiment, and still referring to FIG. 1, probabilistic vector may be determined as a function of a probabilistic machine-learning model. As used in this disclosure a “probabilistic machine-learning model” is a machine-learning model to produce a probabilistic vector output given adrenal enumerations as inputs; this is in contrast to a non-machine learning software program where the commands to be executed are determined in advance by a user and written in a programming language. Probabilistic machine-learning model may include one or more probabilistic machine-learning processes such as supervised, unsupervised, or reinforcement machine-learning processes that computing device 104 and/or a remote device may or may not use in the determination of probabilistic vector, wherein a remote device is an external device to computing device 104 as described above in detail. A probabilistic machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train probabilistic machine-learning process as a function of a probabilistic training set. As used in this disclosure a “probabilistic training set” is a training set that correlates at least an adrenal enumeration to a probabilistic vector. For example, and without limitation, an adrenal enumeration of 34 for reduced cortisol secretion may relate to a probabilistic vector of 85 for the probability of developing an adrenal gland dysregulation. The probabilistic training set may be received as a function of user-entered valuations of adrenal enumerations, and/or probabilistic vectors. Computing device 104 may receive probabilistic training set by receiving correlations of adrenal enumerations and/or probabilistic vectors that were previously received and/or determined during a previous iteration of determining probabilistic vectors. The probabilistic training set may be received by one or more remote devices that at least correlate an adrenal enumeration to a probabilistic vector, wherein a remote device is an external device to computing device 104, as described above. Probabilistic training set may be received in the form of one or more user-entered correlations of an adrenal enumeration to a probabilistic vector. Additionally or alternatively, a user may include an informed advisor, wherein an informed advisor may include, without limitation, family physicians, endocrinologists, gastroenterologists, internists, oncologists, pediatricians, cardiologists, geneticists, neurologists, physical therapists, primary care providers, and the like thereof.

Still referring to FIG. 1, computing device 104 may receive probabilistic machine-learning model from a remote device that utilizes one or more probabilistic machine learning processes, wherein remote device is described above in detail. For example, and without limitation, remote device may include a computing device, external device, processor, and the like thereof. Remote device may perform the probabilistic machine-learning process using the probabilistic training set to generate probabilistic vector and transmit the output to computing device 104. Remote device may transmit a signal, bit, datum, or parameter to computing device 104 that at least relates to probabilistic vector. Additionally or alternatively, the remote device may provide an updated machine-learning model. For example, and without limitation, an updated machine-learning model may be comprised of a firmware update, a software update, a probabilistic machine-learning process correction, and the like thereof. As a non-limiting example a software update may incorporate a probabilistic vector that relates to a modified adrenal enumeration. Additionally or alternatively, the updated machine learning model may be transmitted to the remote device, wherein the remote device may replace the probabilistic machine-learning model with the updated machine-learning model and determine the probabilistic vector as a function of the adrenal enumeration using the updated machine-learning model. The updated machine-learning model may be transmitted by the remote device and received by computing device 104 as a software update, firmware update, or corrected probabilistic machine-learning model. For example, and without limitation a probabilistic machine-learning model may utilize a neural net machine-learning process, wherein the updated machine-learning model may incorporate polynomial regression machine-learning process. Updated machine learning model may additionally or alternatively include any machine-learning model used as an updated machine learning model as described in U.S. Nonprovisional application Ser. No. 17/106,658, the entirety of which is incorporated herein by reference. In an embodiment, and without limitation, probabilistic machine-learning model may identify probabilistic vector as a function of one or more classifiers, wherein a classifier is described above in detail.

Still referring to FIG. 1, computing device 104 may produce adrenal profile 128 by identifying an adrenal dysregulation. As used in this disclosure “adrenal dysregulation” is an ailment and/or collection of ailments that impact an individual's adrenal glands. As a non-limiting example, adrenal dysregulation may include adrenal cancer, adrenal incidentaloma, Addison's disease, Cushing's disease, pheochromocytoma, Conn's syndrome, congenital adrenal hyperplasia, overactive adrenal glands, hyperaldosteronism, adrenocortical carcinoma, pituitary tumors, adrenal gland suppression, and the like thereof. Adrenal dysregulation may be identified as a function of one or more dysregulation machine-learning models. As used in this disclosure “dysregulation machine-learning model” is a machine-learning model to produce an adrenal dysregulation output given adrenal enumerations as inputs; this is in contrast to a non-machine learning software program where the commands to be executed are determined in advance by a user and written in a programming language. Dysregulation machine-learning model may include one or more dysregulation machine-learning processes such as supervised, unsupervised, or reinforcement machine-learning processes that computing device 104 and/or a remote device may or may not use in the determination of adrenal dysregulation. A dysregulation machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train dysregulation machine-learning process as a function of a dysregulation training set. As used in this disclosure “dysregulation training set” is a training set that correlates at least a health system effect and adrenal enumeration 112 to an adrenal dysregulation. As used in this disclosure “health system effect” is an impact and/or effect on the health system of an individual. For example, and without limitation, a health system effect may include fatty deposits, muscle weakness, purple stretch marks, and the like thereof. As a non-limiting example an adrenal enumeration of 71 and a health system effect of reduced cortisol production may relate to an adrenal dysregulation of Addison's disease. The dysregulation training set may be received as a function of user-entered valuations of adrenal enumerations, health system effects, and/or adrenal dysregulations. Computing device 104 may receive dysregulation training by receiving correlations of adrenal enumerations and/or health system effects that were previously received and/or determined during a previous iteration of determining adrenal dysregulations. The dysregulation training set may be received by one or more remote devices that at least correlate adrenal enumerations and/or health system effects to adrenal dysregulations, wherein a remote device is an external device to computing device 104, as described above. The dysregulation training set may be received by one or more user-entered correlations of adrenal enumerations and health system effects to adrenal dysregulations. Additionally or alternatively, a user may include an informed advisor, wherein an informed advisor may include, without limitation, family physicians, endocrinologists, gastroenterologists, internists, oncologists, pediatricians, cardiologists, geneticists, neurologists, physical therapists, primary care providers, and the like thereof.

Still referring to FIG. 1, computing device 104 may receive dysregulation machine-learning model from a remote device that utilizes one or more dysregulation machine learning processes, wherein a remote device is described above in detail. For example, and without limitation, a remote device may include a computing device, external device, processor, and the like thereof. Remote device may perform the dysregulation machine-learning process using the dysregulation training set to generate adrenal dysregulation and transmit the output to computing device 104. Remote device may transmit a signal, bit, datum, or parameter to computing device 104 that at least relates to adrenal dysregulations. Additionally or alternatively, the remote device may provide an updated machine-learning model. For example, and without limitation, an updated machine-learning model may be comprised of a firmware update, a software update, a dysregulation machine-learning process correction, and the like thereof. As a non-limiting example a software update may incorporate a new adrenal enumeration that relates to a modified health system effect. Additionally or alternatively, the updated machine learning model may be transmitted to the remote device, wherein the remote device may replace the dysregulation machine-learning model with the updated machine-learning model and determine the adrenal dysregulation as a function of the adrenal enumeration using the updated machine-learning model. The updated machine-learning model may be transmitted by the remote device and received by computing device 104 as a software update, firmware update, or corrected dysregulation machine-learning model. For example, and without limitation dysregulation machine-learning model may utilize a neural net machine-learning process, wherein the updated machine-learning model may incorporate hierarchical clustering machine-learning process.

In an embodiment, and still referring to FIG. 1, computing device 104 may identify adrenal profile 128 by determining an autoimmune indicator. As used in this disclosure an “autoimmune indicator” is an element of data that denotes a determination and/or likelihood that an individual's immune system is attacking and/or destroying healthy body tissues by mistake. Autoimmune indicator may include one or more indicators such as HLA-B27, HLA-DR4, HLA-DR3, and the like thereof. As a further non-limiting example, autoimmune indicator may include one or more indicators such as, but not limited to, hemoglobin A1c (HbA1c), red blood cell magnesium, serum magnesium, complete blood count red blood cell count, white blood cell count, vitamin D, ferritin, cortisol, high sensitivity C reactive protein (hsCRP), alanine aminotransferase (ALT), glucose, hemoglobin A1c, DHEAS, and/or testosterone. Autoimmune indicator may include any autoimmune indicator used as an autoimmune indicator as described in U.S. Nonprovisional application Ser. No. 17/007,318, filed on Aug. 31, 2020, and entitled “SYSTEM AND METHOD FOR REPRESENTING AN ARRANGED LIST OF PROVIDER ALIMENT POSSIBILITIES,” the entirety of which is incorporated herein by reference.

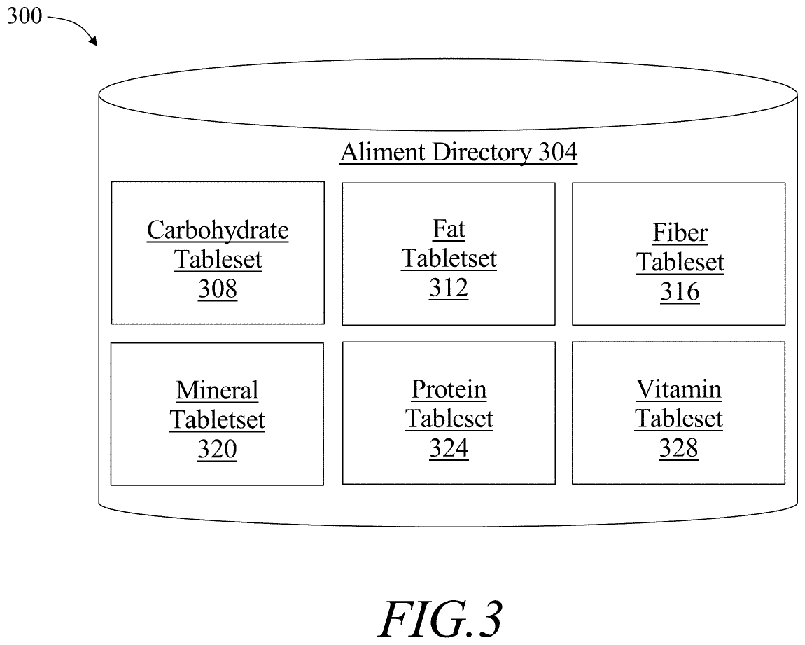

Still referring to FIG. 1, computing device 104 determines an edible 140 as a function of adrenal profile 128. As used in this disclosure an “edible” is a source of nourishment that may be consumed by a user such that the user may absorb the nutrients from the source. For example and without limitation, an edible may include legumes, plants, fungi, nuts, seeds, breads, dairy, eggs, meat, cereals, rice, seafood, desserts, dried foods, dumplings, pies, noodles, salads, stews, soups, sauces, sandwiches, and the like thereof. In an embodiment, edible may be determined as a function of physiological response, wherein a first edible may be identified for a first physiological response parameter and a second edible may be determined for a second physiological response. For example, and without limitation, a first edible of bananas may be determined for a first physiological response of diarrhea, wherein a second edible of steak may be identified for a second physiological response of high blood sugar. Computing device 104 may determine edible 140 as a function of receiving a nourishment composition. As used in this disclosure a “nourishment composition” is a list and/or compilation of all of the nutrients contained in an edible. As a non-limiting example nourishment composition may include one or more quantities and/or amounts of total fat, including saturated fat and/or trans-fat, cholesterol, sodium, total carbohydrates, including dietary fiber and/or total sugars, protein, vitamin A, vitamin C, thiamin, riboflavin, niacin, pantothenic acid, vitamin b6, folate, biotin, vitamin B12, vitamin D, vitamin E, vitamin K, calcium, iron, phosphorous, iodine, magnesium, zinc, selenium, copper, manganese, chromium, molybdenum, chloride, and the like thereof. Nourishment composition may be obtained as a function of an edible directory, wherein an “edible directory” is a database of edibles that may be identified as a function of one or more metabolic components, as described in detail below, in reference to FIG. 3.

Still referring to FIG. 1, computing device 104 may produce a nourishment desideration as a function of adrenal profile 128. As used in this disclosure a “nourishment desideration” is requirement and/or necessary amount of nutrients required for a user to consume. As a non-limiting example, nourishment desideration may include a user requirement of 5 mg of vitamin B5 to be consumed per day. Nourishment desideration may be determined as a function of receiving a nourishment goal. As used in this disclosure a “nourishment goal” is a recommended amount of nutrients that a user should consume. Nourishment goal may be identified by one or more organizations that relate to, represent, and/or study adrenal glands in humans, such as the American Medical Association, National Organization for Rare Diseases, National Adrenal Diseases Foundation, Adrenal Health and Disease, Endocrine Society, Hormone Health Network, American Association of Clinical Endocrinology, and the like thereof.

Still referring to FIG. 1, computing device 104 identifies edible 140 as a function of nourishment composition, nourishment desideration, and an edible machine-learning model. As used in this disclosure a “edible machine-learning model” is a machine-learning model to produce an edible output given nourishment compositions and nourishment desiderations as inputs; this is in contrast to a non-machine learning software program where the commands to be executed are determined in advance by a user and written in a programming language. Edible machine-learning model may include one or more edible machine-learning processes such as supervised, unsupervised, or reinforcement machine-learning processes that computing device 104 and/or a remote device may or may not use in the determination of edible 140, wherein a remote device is an external device to computing device 104 as described above in detail. An edible machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train edible machine-learning process as a function of an edible training set. As used in this disclosure an “edible training set” is a training set that correlates at least nourishment composition and nourishment desideration to an edible. For example, and without limitation, nourishment composition of 200 g of carbohydrates and a nourishment desideration of 175 g of carbohydrates as a function of an adrenal dysregulation pheochromocytoma may relate to an edible of bread. The edible training set may be received as a function of user-entered valuations of nourishment compositions, nourishment desiderations, and/or edibles. Computing device 104 may receive edible training set by receiving correlations of nourishment compositions and/or nourishment desiderations that were previously received and/or determined during a previous iteration of determining edibles. The edible training set may be received by one or more remote devices that at least correlate a nourishment composition and nourishment desideration to an edible, wherein a remote device is an external device to computing device 104, as described above. Edible training set may be received in the form of one or more user-entered correlations of a nourishment composition and/or nourishment desideration to an edible. Additionally or alternatively, a user may include an informed advisor, wherein an informed advisor may include, without limitation family physicians, endocrinologists, gastroenterologists, internists, oncologists, pediatricians, cardiologists, geneticists, neurologists, physical therapists, primary care providers, and the like thereof.

Still referring to FIG. 1, computing device 104 may receive edible machine-learning model from a remote device that utilizes one or more edible machine learning processes, wherein remote device is described above in detail. For example, and without limitation, remote device may include a computing device, external device, processor, and the like thereof. Remote device may perform the edible machine-learning process using the edible training set to generate edible 140 and transmit the output to computing device 104. Remote device may transmit a signal, bit, datum, or parameter to computing device 104 that at least relates to edible 140. Additionally or alternatively, the remote device may provide an updated machine-learning model. For example, and without limitation, an updated machine-learning model may be comprised of a firmware update, a software update, an edible machine-learning process correction, and the like thereof. As a non-limiting example a software update may incorporate a new nourishment composition that relates to a modified nourishment desideration. Additionally or alternatively, the updated machine learning model may be transmitted to the remote device, wherein the remote device may replace the edible machine-learning model with the updated machine-learning model and determine the edible as a function of the nourishment desideration using the updated machine-learning model. The updated machine-learning model may be transmitted by the remote device and received by computing device 104 as a software update, firmware update, or corrected edible machine-learning model. For example, and without limitation an edible machine-learning model may utilize a neural net machine-learning process, wherein the updated machine-learning model may incorporate polynomial regression machine-learning process. Updated machine learning model may additionally or alternatively include any machine-learning model used as an updated machine learning model as described in U.S. Nonprovisional application Ser. No. 17/106,658, filed on Nov. 30, 2020, and entitled “A SYSTEM AND METHOD FOR GENERATING A DYNAMIC WEIGHTED COMBINATION,” the entirety of which is incorporated herein by reference. In an embodiment, and without limitation, edible machine-learning model may identify edible 140 as a function of one or more classifiers, wherein a classifier is described above in detail.

Still referring to FIG. 1, computing device 104 may identify edible as a function of a likelihood parameter. As used in this disclosure a “likelihood parameter” is a parameter that identities the probability of a user to consume an edible. As a non-limiting example likelihood parameter may identify a high probability that a user will consume an edible of chicken. As a further non-limiting example likelihood parameter may identify a low probability that a user will consume an edible of spinach. Likelihood parameter may be determined as a function of a user taste profile. As used in this disclosure a “user taste profile” is a profile of a user that identifies one or more desires, preferences, wishes, and/or wants that a user has. As a non-limiting example a user taste profile may include a user's preference for chocolate flavor and/or hard textured edibles. Likelihood parameter may be determined as a function of an edible profile. As used in this disclosure an “edible profile” is taste of an edible is the sensation of flavor perceived in the mouth and throat on contact with the edible. Edible profile may include one or more flavor variables. As used in this disclosure a “flavor variable” is a variable associated with the distinctive taste of an edible, wherein a distinctive may include, without limitation sweet, bitter, sour, salty, umami, cool, and/or hot. Edible profile may be determined as a function of receiving flavor variable from a flavor directory. As used in this disclosure a “flavor directory” is a database or other data structure including flavors for an edible. As a non-limiting example flavor directory may include a list and/or collection of edibles that all contain salty flavor variables. As a further non-limiting example flavor directory may include a list and/or collection of edibles that all contain sour flavor variables. Flavor directory may be implemented similarly to an edible directory as described below in detail, in reference to FIG. 3. Likelihood parameter may alternatively or additionally include any user taste profile and/or edible profile used as a likelihood parameter as described in U.S. Nonprovisional application Ser. No. 17/032,080, filed on Sep. 25, 2020, and entitled “METHODS, SYSTEMS, AND DEVICES FOR GENERATING A REFRESHMENT INSTRUCTION SET BASED ON INDIVIDUAL PREFERENCES,” the entirety of which is incorporated herein by reference.

Still referring to FIG. 1, computing device 104 generates a nourishment program 144 as a function of edible 140. As used in this disclosure a “nourishment program” is a program consisting of one or more edibles that are to be consumed over a given time period, wherein a time period is a temporal measurement such as seconds, minutes, hours, days, weeks, months, years, and the like thereof. As a non-limiting example nourishment program 144 may consist of recommending steak for 8 days. As a further non-limiting example nourishment program 144 may recommend fish for a first day, legumes for a second day, and spinach for a third day. Nourishment program 144 may include one or more diet programs such as paleo, keto, vegan, vegetarian, Mediterranean, Dukan, Zone, HCG, and the like thereof. Computing device 104 may develop nourishment program 144 as a function of an intended outcome. As used in this disclosure an “intended outcome” is an outcome that an edible may generate according to a predicted and/or purposeful plan. As a non-limiting example, intended outcome may include a treatment outcome. As used in this disclosure a “treatment outcome” is an intended outcome that is designed to at least reverse and/or eliminate adrenal profile 128, adrenal movement 132, and/or adrenal dysregulation. As a non-limiting example, a treatment outcome may include reversing the effects of the adrenal dysregulation adrenal insufficiency. As a further non-limiting example, a treatment outcome includes reversing the adrenal dysregulation hyperaldosteronism. Intended outcome may include a prevention outcome. As used in this disclosure a “prevention outcome” is an intended outcome that is designed to at least prevent and/or avert adrenal profile 128, adrenal movement 132, and/or adrenal dysregulation. As a non-limiting example, a prevention outcome may include preventing the development of the adrenal dysregulation congenital adrenal hyperplasia.

Still referring to FIG. 1, computing device 104 may develop nourishment program 144 as a function of edible 140 and intended outcome using a nourishment machine-learning model. As used in this disclosure a “nourishment machine-learning model” is a machine-learning model to produce a nourishment program output given edibles and/or intended outcomes as inputs; this is in contrast to a non-machine learning software program where the commands to be executed are determined in advance by a user and written in a programming language. Nourishment machine-learning model may include one or more nourishment machine-learning processes such as supervised, unsupervised, or reinforcement machine-learning processes that computing device 104 and/or a remote device may or may not use in the development of nourishment program 144. Nourishment machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train nourishment machine-learning process as a function of a nourishment training set. As used in this disclosure a “nourishment training set” is a training set that correlates an intended outcome to an edible. The nourishment training set may be received as a function of user-entered edibles, intended outcomes, and/or nourishment programs. For example, and without limitation, an intended outcome of treating Cushing Syndrome may correlate to an edible of blueberries. Computing device 104 may receive nourishment training by receiving correlations of intended outcomes and/or edibles that were previously received and/or determined during a previous iteration of developing nourishment programs. The nourishment training set may be received by one or more remote devices that at least correlate an intended outcome and/or edible to a nourishment program, wherein a remote device is an external device to computing device 104, as described above. Nourishment training set may be received in the form of one or more user-entered correlations of an intended outcome and/or edible to a nourishment program. Additionally or alternatively, a user may include an informed advisor, wherein an informed advisor may include, without limitation family physicians, endocrinologists, gastroenterologists, internists, oncologists, pediatricians, cardiologists, geneticists, neurologists, physical therapists, primary care providers, and the like thereof.

Still referring to FIG. 1, computing device 104 may receive nourishment machine-learning model from the remote device that utilizes one or more nourishment machine learning processes, wherein a remote device is described above in detail. For example, and without limitation, a remote device may include a computing device, external device, processor, and the like thereof. The remote device may perform the nourishment machine-learning process using the nourishment training set to develop nourishment program 144 and transmit the output to computing device 104. The remote device may transmit a signal, bit, datum, or parameter to computing device 104 that at least relates to nourishment program 144. Additionally or alternatively, the remote device may provide an updated machine-learning model. For example, and without limitation, an updated machine-learning model may be comprised of a firmware update, a software update, a nourishment machine-learning process correction, and the like thereof. As a non-limiting example a software update may incorporate a new intended outcome that relates to a modified edible. Additionally or alternatively, the updated machine learning model may be transmitted to the remote device, wherein the remote device may replace the nourishment machine-learning model with the updated machine-learning model and develop the nourishment program as a function of the intended outcome using the updated machine-learning model. The updated machine-learning model may be transmitted by the remote device and received by computing device 104 as a software update, firmware update, or corrected nourishment machine-learning model. For example, and without limitation nourishment machine-learning model may utilize a neural net machine-learning process, wherein the updated machine-learning model may incorporate decision tree machine-learning processes.

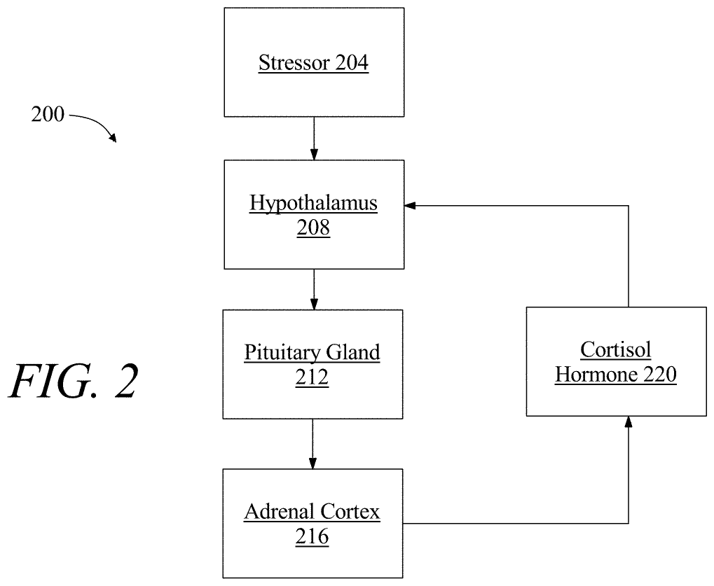

Now referring to FIG. 2, an exemplary embodiment 200 of a homeostasis is illustrated. In an embodiment homeostasis may include a stressor 204. As used in this disclosure a “stressor” is an action, entity, and/or object that causes an individual stress, strain and/or tension. For example, and without limitation, a stressor may include public speaking, working long hours, poor management, discrimination, harassment, fear of uncertainty, death, divorce, loss of job, marriage, chronic illness, injury, emotional problems, traumatic events, moving to a new home, financial struggles, major life changes, and the like thereof. Stressor 204 may stimulate a hypothalamus 208. As used in this disclosure a “hypothalamus” is a region of the forebrain below the thalamus. In an embodiment hypothalamus may secret one or more hormones that control and/or regulate one or more glands. For example, and without limitation hypothalamus may include a region that secretes corticotropin-releasing hormone to innervate a pituitary gland 212. As used in this disclosure a “pituitary gland” is an endocrine gland located below the hypothalamus. In an embodiment pituitary gland 212 may synthesize and/or secret hormones to target organs and/or tissues in the human body. For example, and without limitation pituitary gland 212 may secrete one or more hormones such as somatotropes, corticotropes, thyrotropes, gonadotropes, lactotropes, and the like thereof. In an embodiment and without limitation, pituitary gland 212 may secrete adrenocorticotropic hormone to an adrenal cortex 216. As used in this disclosure an “adrenal cortex” is the outer region of the adrenal gland. Adrenal cortex 216 may be composed of three separate zones, such as but not limited to the zona glomerulosa, zona fasciculata, zona reticularis, and the like thereof. In an embodiment, and without limitation, adrenal cortex 216 may produce a cortisol hormone 220. As used in this disclosure a “cortisol hormone” is a steroid hormone in the human body. Cortisol hormone 220 may be produced as a function of the zona fasciculata region of adrenal cortex 216. Cortisol hormone 220 may include one or more functions to increase blood sugar, suppress the immune system, aid in metabolizing fat, protein, and/or carbohydrates, decrease bone formation, and the like thereof. In an embodiment, and without limitation homeostasis may include a negative feedback loop. As used in this disclosure a “negative feedback loop” is a feedback system wherein an output signals to the process and/or mechanism to stop producing the output. For example, and without limitation, cortisol hormone 220 may signal to hypothalamus 208 to reduce the production and secretion of corticotropin-releasing hormone of hypothalamus 208, wherein the reduced production and secretion of corticotropin-releasing hormone also reduces production of adrenocorticotropic hormone and subsequently the production of cortisol hormone.

Now referring to FIG. 3, an exemplary embodiment 300 of an edible directory 304 according to an embodiment of the invention is illustrated. Edible directory 304 may be implemented, without limitation, as a relational databank, a key-value retrieval databank such as a NOSQL databank, or any other format or structure for use as a databank that a person skilled in the art would recognize as suitable upon review of the entirety of this disclosure. Edible directory 304 may alternatively or additionally be implemented using a distributed data storage protocol and/or data structure, such as a distributed hash table or the like. Edible directory 304 may include a plurality of data entries and/or records as described above. Data entries in a databank may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a databank may store, retrieve, organize, and/or reflect data and/or records as used herein, as well as categories and/or populations of data consistently with this disclosure. Edible directory 304 may include a carbohydrate tableset 308. Carbohydrate tableset 308 may relate to a nourishment composition of an edible with respect to the quantity and/or type of carbohydrates in the edible. As a non-limiting example, carbohydrate tableset 308 may include monosaccharides, disaccharides, oligosaccharides, polysaccharides, and the like thereof. Edible directory 304 may include a fat tableset 312. Fat tableset 312 may relate to a nourishment composition of an edible with respect to the quantity and/or type of esterified fatty acids in the edible. Fat tableset 312 may include, without limitation, triglycerides, monoglycerides, diglycerides, phospholipids, sterols, waxes, and free fatty acids. Edible directory 304 may include a fiber tableset 316. Fiber tableset 316 may relate to a nourishment composition of an edible with respect to the quantity and/or type of fiber in the edible. As a non-limiting example, fiber tableset 316 may include soluble fiber, such as beta-glucans, raw guar gum, psyllium, inulin, and the like thereof as well as insoluble fiber, such as wheat bran, cellulose, lignin, and the like thereof. Edible directory 304 may include a mineral tableset 320. Mineral tableset 320 may relate to a nourishment composition of an edible with respect to the quantity and/or type of minerals in the edible. As a non-limiting example, mineral tableset 320 may include calcium, phosphorous, magnesium, sodium, potassium, chloride, sulfur, iron, manganese, copper, iodine, zing, cobalt, fluoride, selenium, and the like thereof. Edible directory 304 may include a protein tableset 324. Protein tableset 324 may relate to a nourishment composition of an edible with respect to the quantity and/or type of proteins in the edible. As a non-limiting example, protein tableset 324 may include amino acids combinations, wherein amino acids may include, without limitation, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and the like thereof. Edible directory 304 may include a vitamin tableset 328. Vitamin tableset 328 may relate to a nourishment composition of an edible with respect to the quantity and/or type of vitamins in the edible. As a non-limiting example, vitamin tableset 328 may include vitamin A, vitamin B₁, vitamin B₂, vitamin B₃, vitamin B₅, vitamin B₆, vitamin B₇, vitamin B₉, vitamin B₁₂, vitamin C, vitamin D, vitamin E, vitamin K, and the like thereof.

Now referring to FIG. 4, an exemplary embodiment 400 of adrenal movement 132 is illustrated. Adrenal movement 132 may be determined as a function of plotting adrenal enumeration 112 on a y-axis and a medical examination 404 on the x-axis. As used in this disclosure a “medical examination” is an evaluation and/or estimation of the individual's health system. Medical examination may include one or more examinations that identifies, characterizes, and/or otherwise evaluates the adrenal glands of the endocrine system of an individual. In an embodiment, and without limitation, medical examination may include a southern blot examination, a polymerase chain reaction examination, a genetic sequencing examination, a mass spectrometry examination, a blood test, a urine test, a biopsy, a CT scan, an Mill, a Metaiodobenzylguanidine scan, an adrenal vein sampling, and the like thereof. Medical examination may be conducted by one or more informed advisors. As used in this disclosure “informed advisor” is an individual that is skilled in an area relating to the study of the health system of the individual. As a non-limiting example an informed advisor may include a medical professional who may assist and/or participate in the medical diagnosis, treatment, and/or guidance of an individual's health system including, but not limited to, family physicians, endocrinologists, gastroenterologists, internists, oncologists, pediatricians, cardiologists, geneticists, neurologists, physical therapists, primary care providers, and the like thereof. As a non-limiting example input may include an informed advisor that enters a medical examination comprising a blood analysis, urine analysis, stool analysis, saliva analysis, skin analysis, and the like thereof. Additionally or alternatively, input may include one or more medical records and/or patient charts that identify an individual's previous medical history. In an embodiment, and without limitation, x-y plot may plot a first adrenal enumeration as a function of a first medical examination and a second adrenal enumeration as a function of a second medical examination. For example, and without limitation, a first adrenal enumeration of 5 may be plotted for a first medical examination of a blood analysis, wherein a second adrenal enumeration of 12 may be plotted for a second medical examination of a stool analysis. First medical examination and second examination may be the same type and/or style of examination. For example, the first medical examination may be urine sample, wherein the second medical examination may also be a urine sample collected at one or more different time periods, wherein a time period is a period of time such as seconds, minutes, hours, days, weeks, months, years, and the like thereof.

Still referring to FIG. 4, adrenal movement 132 may include determining an upper limit 408 as a function of the x-y plot. As used in this disclosure an “upper limit” is a statistical limit that an adrenal enumeration may not exceed for a plurality of medical examinations. For example, and without limitation upper limit 408 may include a standard deviation maximum, a natural process limit, a maximum probability distribution, and the like thereof. As a further non-limiting example, upper limit 408 may include a limit that an adrenal enumeration may not be 3 standard deviations greater than a mean, range, proportion, probability, and the like thereof. Adrenal movement 132 may include determining an upper control 412 as a function of the x-y plot. As used in this disclosure an “upper control” is a statistical warning that an adrenal enumeration may be diverging above a mean for a plurality of medical examinations. For example, and without limitation upper control 412 may include a standard deviation range, a natural process range, a probability distribution, and the like thereof. As a further non-limiting example, upper control 412 may include a statistical warning that an adrenal enumeration may not be 2 standard deviations greater than a mean, range, proportion, probability, and the like thereof. Adrenal movement 132 may include determining a lower control 416 as a function of the x-y plot. As used in this disclosure an “lower control” is a statistical warning that an adrenal enumeration may be diverging below a mean for a plurality of medical examinations. For example, and without limitation lower control 416 may include a standard deviation range, a natural process range, a probability distribution, and the like thereof. As a further non-limiting example, lower control 416 may include a statistical warning that an adrenal enumeration may not be 2 standard deviations inferior to a mean, range, proportion, probability, and the like thereof. Adrenal movement 132 may include determining a lower limit 420 as a function of the x-y plot. As used in this disclosure an “lower limit” is a statistical limit that an adrenal enumeration may not be inferior to for a plurality of medical examinations. For example, and without limitation lower limit 420 may include a standard deviation minimum, a natural process limit, a minimum probability distribution, and the like thereof. As a further non-limiting example, lower limit 420 may include a limit that an adrenal enumeration may not be 3 standard deviations below a mean, range, proportion, probability, and the like thereof.

In an embodiment, and still referring to FIG. 4, adrenal movement 132 may be determined as a function of one or more rule sets. As used in this disclosure a “rule set” is a set of guidelines and/or rules to be followed to detect a signal and/or trend of the adrenal movement. For example, and without limitation, rule set may include identifying a malfunction and/or dysregulation as a function of an adrenal enumeration that exceed and/or is inferior to upper limit 408 and/or lower limit 420. In an embodiment, rule set may include identifying n number of consecutive adrenal enumerations that are all above and/or below a central mean, median, and/or mode of the x-y plot. For example, 7 consecutive adrenal enumerations above the mean of the x-y plot may indicate a trend in the adrenal function such as overproduction of one or more mineralocorticoids. In yet another embodiment, rule set may include identifying n number of consecutive adrenal enumerations that are all increasing and/or decreasing in the x-y plot. For example, 5 consecutive adrenal enumerations that are all decreasing may indicate a negative trend in the adrenal function such as decreased regulation of cortisol in the circulatory system.

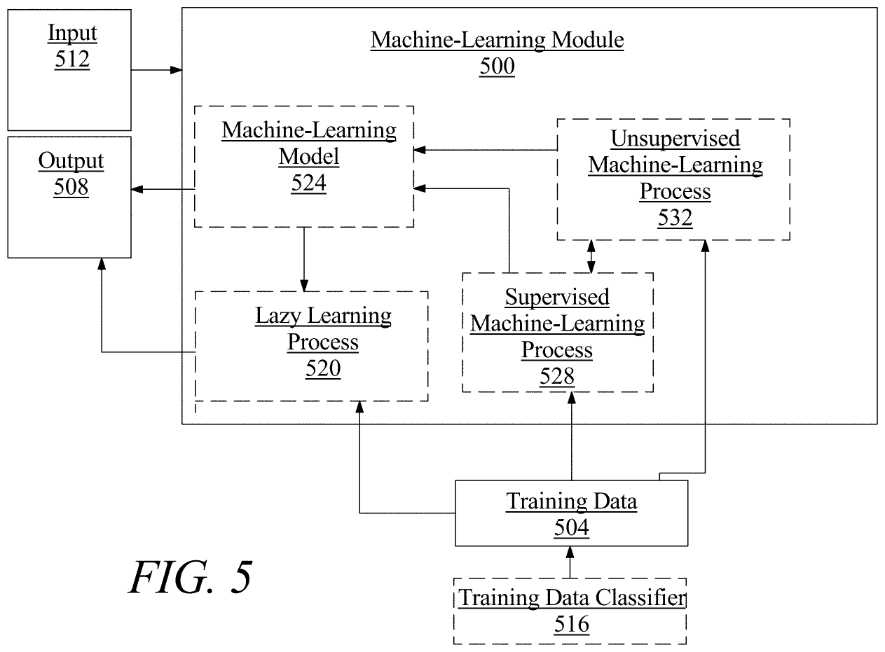

Referring now to FIG. 5, an exemplary embodiment of a machine-learning module 500 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 504 to generate an algorithm that will be performed by a computing device/module to produce outputs 508 given data provided as inputs 512; this is in contrast to a non-machine learning software program where the commands to be executed are determined in advance by a user and written in a programming language.

Still referring to FIG. 5, “training data,” as used herein, is data containing correlations that a machine-learning process may use to model relationships between two or more categories of data elements. For instance, and without limitation, training data 504 may include a plurality of data entries, each entry representing a set of data elements that were recorded, received, and/or generated together; data elements may be correlated by shared existence in a given data entry, by proximity in a given data entry, or the like. Multiple data entries in training data 504 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 504 according to various correlations; correlations may indicate causative and/or predictive links between categories of data elements, which may be modeled as relationships such as mathematical relationships by machine-learning processes as described in further detail below. Training data 504 may be formatted and/or organized by categories of data elements, for instance by associating data elements with one or more descriptors corresponding to categories of data elements. As a non-limiting example, training data 504 may include data entered in standardized forms by persons or processes, such that entry of a given data element in a given field in a form may be mapped to one or more descriptors of categories. Elements in training data 504 may be linked to descriptors of categories by tags, tokens, or other data elements; for instance, and without limitation, training data 504 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 5, training data 504 may include one or more elements that are not categorized; that is, training data 504 may not be formatted or contain descriptors for some elements of data. Machine-learning algorithms and/or other processes may sort training data 504 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 504 to be made applicable for two or more distinct machine-learning algorithms as described in further detail below. Training data 504 used by machine-learning module 500 may correlate any input data as described in this disclosure to any output data as described in this disclosure. As a non-limiting illustrative example an input of an adrenal movement and/or adrenal enumeration may result in an adrenal profile output.

Further referring to FIG. 5, training data may be filtered, sorted, and/or selected using one or more supervised and/or unsupervised machine-learning processes and/or models as described in further detail below; such models may include without limitation a training data classifier 516. Training data classifier 516 may include a “classifier,” which as used in this disclosure is a machine-learning model as defined below, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 500 may generate a classifier using a classification algorithm, defined as a processes whereby a computing device and/or any module and/or component operating thereon derives a classifier from training data 504. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 516 may classify elements of training data to sub-categories of adrenal movements, such as positive trends, negative trends, and the like thereof.

Still referring to FIG. 5, machine-learning module 500 may be configured to perform a lazy-learning process 520 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 504. Heuristic may include selecting some number of highest-ranking associations and/or training data 504 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 5, machine-learning processes as described in this disclosure may be used to generate machine-learning models 524. A “machine-learning model,” as used in this disclosure, is a mathematical and/or algorithmic representation of a relationship between inputs and outputs, as generated using any machine-learning process including without limitation any process as described above, and stored in memory; an input is submitted to a machine-learning model 524 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 524 may be generated by creating an artificial neural network, such as a convolutional neural network comprising an input layer of nodes, one or more intermediate layers, and an output layer of nodes. Connections between nodes may be created via the process of “training” the network, in which elements from a training data 504 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 5, machine-learning algorithms may include at least a supervised machine-learning process 528. At least a supervised machine-learning process 528, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include adrenal movements and/or adrenal enumerations as described above as inputs, adrenal profiles as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 504. Persons skilled in the art, upon reviewing the entirety of this disclosure, will be aware of various possible variations of at least a supervised machine-learning process 528 that may be used to determine relation between inputs and outputs. Supervised machine-learning processes may include classification algorithms as defined above.

Further referring to FIG. 5, machine learning processes may include at least an unsupervised machine-learning processes 532. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 5, machine-learning module 500 may be designed and configured to create a machine-learning model 524 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 5, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

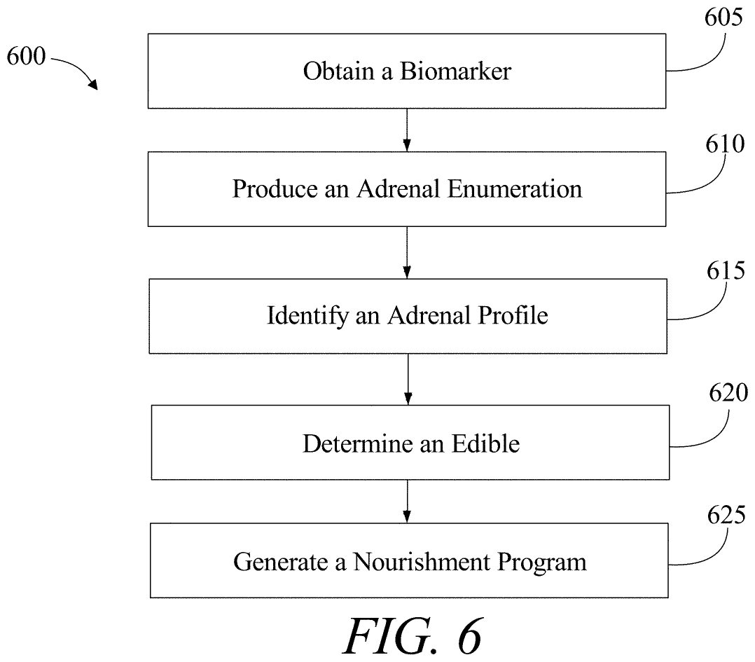

Now referring to FIG. 6, an exemplary embodiment 600 of a method for generating an adrenal dysregulation nourishment program is illustrated. At step 605, a computing device 104 obtains a biomarker 108. Computing device 104 includes any of the computing device 104 as described above, in reference to FIGS. 1-5. Biomarker 108 includes any of the biomarker 108 as described above, in reference to FIGS. 1-5.

Still referring to FIG. 6, at step 610, computing device 104 produces an adrenal enumeration 112 as a function of biomarker 108. Adrenal enumeration 112 includes any of the adrenal enumeration 112 as described above, in reference to FIGS. 1-5. Computing device 104 produces adrenal enumeration 112 as a function of receiving a homeostatic element 116. Homeostatic element 116 includes any of the homeostatic element 116 as described above, in reference to FIGS. 1-5. Computing device 104 produces adrenal enumeration 112 as a function of identifying a homeostatic divergence 120 as a function of biomarker 108 and homeostatic element 116. Homeostatic divergence 120 includes any of the homeostatic divergence 120 as described above, in reference to FIGS. 1-5. Computing device 104 produces adrenal enumeration 112 as a function of homeostatic divergence 120 and a statistical deviation 124. Statistical deviation 124 includes any of the statistical deviation 124 as described above, in reference to FIGS. 1-5.

Still referring to FIG. 6, at step 615, computing device 104 identifies an adrenal profile 128 as a function of adrenal enumeration 112. Adrenal profile 128 includes any of the adrenal profile 128 as described above, in reference to FIGS. 1-5. Computing device 104 identifies adrenal profile 128 as a function of determining an adrenal movement 132. Adrenal movement 132 includes any of the adrenal movement 132 as described above, in reference to FIGS. 1-5. Computing device 104 identifies adrenal profile 128 as a function of adrenal enumeration 112 and adrenal movement 132 using an adrenal machine-learning model 136. Adrenal machine-learning model 136 includes any of the adrenal machine-learning model 136 as described above, in reference to FIGS. 1-5.

Still referring to FIG. 6, at step 620, computing device 104 determines an edible 140 as a function of adrenal profile 128. Edible 140 includes any of the edible 140 as described above, in reference to FIGS. 1-5.

Still referring to FIG. 6, at step 625, computing device 104 generates a nourishment program 144 as a function of edible 140. Nourishment program 144 includes any of the nourishment program 144 as described above, in reference to FIGS. 1-5.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

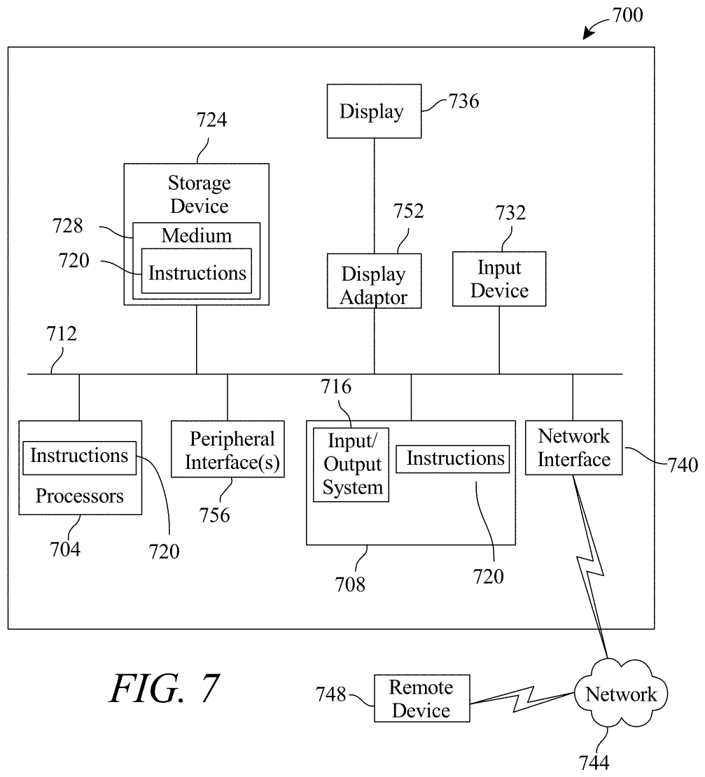

FIG. 7 shows a diagrammatic representation of one embodiment of a computing device in the exemplary form of a computer system 700 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 700 includes a processor 704 and a memory 708 that communicate with each other, and with other components, via a bus 712. Bus 712 may include any of several types of bus structures including, but not limited to, a memory bus, a memory controller, a peripheral bus, a local bus, and any combinations thereof, using any of a variety of bus architectures.

Processor 704 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 704 may be organized according to Von Neumann and/or Harvard architecture as a non-limiting example. Processor 704 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC)

Memory 708 may include various components (e.g., machine-readable media) including, but not limited to, a random-access memory component, a read only component, and any combinations thereof. In one example, a basic input/output system 716 (BIOS), including basic routines that help to transfer information between elements within computer system 700, such as during start-up, may be stored in memory 708. Memory 708 may also include (e.g., stored on one or more machine-readable media) instructions (e.g., software) 720 embodying any one or more of the aspects and/or methodologies of the present disclosure. In another example, memory 708 may further include any number of program modules including, but not limited to, an operating system, one or more application programs, other program modules, program data, and any combinations thereof.

Computer system 700 may also include a storage device 724. Examples of a storage device (e.g., storage device 724) include, but are not limited to, a hard disk drive, a magnetic disk drive, an optical disc drive in combination with an optical medium, a solid-state memory device, and any combinations thereof. Storage device 724 may be connected to bus 712 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 724 (or one or more components thereof) may be removably interfaced with computer system 700 (e.g., via an external port connector (not shown)). Particularly, storage device 724 and an associated machine-readable medium 728 may provide nonvolatile and/or volatile storage of machine-readable instructions, data structures, program modules, and/or other data for computer system 700. In one example, software 720 may reside, completely or partially, within machine-readable medium 728. In another example, software 720 may reside, completely or partially, within processor 704.

Computer system 700 may also include an input device 732. In one example, a user of computer system 700 may enter commands and/or other information into computer system 700 via input device 732. Examples of an input device 732 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 732 may be interfaced to bus 712 via any of a variety of interfaces (not shown) including, but not limited to, a serial interface, a parallel interface, a game port, a USB interface, a FIREWIRE interface, a direct interface to bus 712, and any combinations thereof. Input device 732 may include a touch screen interface that may be a part of or separate from display 736, discussed further below. Input device 732 may be utilized as a user selection device for selecting one or more graphical representations in a graphical interface as described above.

A user may also input commands and/or other information to computer system 700 via storage device 724 (e.g., a removable disk drive, a flash drive, etc.) and/or network interface device 740. A network interface device, such as network interface device 740, may be utilized for connecting computer system 700 to one or more of a variety of networks, such as network 744, and one or more remote devices 748 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 744, may employ a wired and/or a wireless mode of communication. In general, any network topology may be used. Information (e.g., data, software 720, etc.) may be communicated to and/or from computer system 700 via network interface device 740.

Computer system 700 may further include a video display adapter 752 for communicating a displayable image to a display device, such as display device 736. Examples of a display device include, but are not limited to, a liquid crystal display (LCD), a cathode ray tube (CRT), a plasma display, a light emitting diode (LED) display, and any combinations thereof. Display adapter 752 and display device 736 may be utilized in combination with processor 704 to provide graphical representations of aspects of the present disclosure. In addition to a display device, computer system 700 may include one or more other peripheral output devices including, but not limited to, an audio speaker, a printer, and any combinations thereof. Such peripheral output devices may be connected to bus 712 via a peripheral interface 756. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve systems and methods according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A system for generating an adrenal dysregulation nourishment program, the system comprising:
a computing device, the computing device configured to:
obtain a biomarker;
produce an adrenal enumeration as a function of the biomarker; wherein producing the adrenal enumeration further comprises:
receiving a homeostatic element;
identifying a homeostatic divergence as a function of the biomarker and homeostatic element; and
producing the adrenal enumeration as a function of the homeostatic divergence and a statistical deviation;
identify an adrenal profile as a function of the adrenal enumeration, wherein producing the adrenal profile further comprises:
determining an adrenal movement; and
identifying the adrenal profile as a function of the adrenal enumeration and the adrenal movement using an adrenal machine-learning model;
determine an edible as a function of the adrenal profile; and
generate a nourishment program as a function of the edible.

2. The system of claim 1, wherein obtaining a biomarker further comprises receiving a mutation indicator and obtaining the biomarker as a function of the mutation indicator.

3. The system of claim 1, wherein producing the adrenal enumeration further comprises:
determining an origin of malfunction; and
producing the adrenal enumeration as a function of the biomarker and the origin of malfunction using an origin machine-learning model.

4. The system of claim 1, wherein identifying an adrenal profile further comprises determining a physiological alteration and identifying the adrenal profile as a function of the physiological alteration.

5. The system of claim 4, wherein determining the physiological alteration further comprises:
receiving a target function; and
determining the physiological alteration as a function of the target function and adrenal enumeration using a physiological machine-learning model.

6. The system of claim 1, wherein the homeostatic element includes a status of homeostasis.

7. The system of claim 1, wherein identifying the homeostatic divergence further comprises receiving a divergence threshold and identifying the homeostatic divergence as a function of the divergence threshold.

8. The system of claim 1, wherein identifying the adrenal profile includes determining an adrenal dysregulation and producing the adrenal profile as a function of the adrenal dysregulation.

9. The system of claim 1, wherein determining the edible further comprises:
receiving a nourishment composition from an edible directory;
producing a nourishment desideration as a function of the adrenal profile; and
determining the edible as a function of the nourishment composition and the nourishment desideration using an edible machine-learning model.

10. The system of claim 1, wherein generating the nourishment program further comprises:
receiving an intended outcome; and
generating the nourishment program as a function of the intended outcome using a nourishment machine-learning model.

11. A method for generating an adrenal dysregulation nourishment program, the method comprising:
obtaining, by a computing device, a biomarker;
producing, by the computing device, an adrenal enumeration as a function of the biomarker;
wherein producing the adrenal enumeration further comprises:
receiving a homeostatic element;
identifying a homeostatic divergence as a function of the biomarker and homeostatic element; and
producing the adrenal enumeration as a function of the homeostatic divergence and a statistical deviation;
identifying, by the computing device, an adrenal profile as a function of the adrenal enumeration, wherein producing the adrenal profile further comprises:
determining an adrenal movement; and
identifying the adrenal profile as a function of the adrenal enumeration and the adrenal movement using an adrenal machine-learning model;
determining, by the computing device, an edible as a function of the adrenal profile; and
generating, by the computing device, a nourishment program as a function of the edible.

12. The method of claim 11, wherein obtaining a biomarker further comprises receiving a mutation indicator and obtaining the biomarker as a function of the mutation indicator.

13. The method of claim 11, wherein producing the adrenal enumeration further comprises:
determining an origin of malfunction; and
producing the adrenal enumeration as a function of the biomarker and the origin of malfunction using an origin machine-learning model.

14. The method of claim 11, wherein identifying an adrenal profile further comprises determining a physiological alteration and identifying the adrenal profile as a function of the physiological alteration.

15. The method of claim 14, wherein determining the physiological alteration further comprises:
receiving a target function; and
determining the physiological alteration as a function of the target function and adrenal enumeration using a physiological machine-learning model.

16. The method of claim 11, wherein the homeostatic element includes a status of homeostasis.

17. The method of claim 11, wherein identifying the homeostatic divergence further comprises receiving a divergence threshold and identifying the homeostatic divergence as a function of the divergence threshold.

18. The method of claim 11, wherein identifying the adrenal profile includes determining an adrenal dysregulation and producing the adrenal profile as a function of the adrenal dysregulation.

19. The method of claim 11, wherein determining the edible further comprises:
receiving a nourishment composition from an edible directory;
producing a nourishment desideration as a function of the adrenal profile; and
determining the edible as a function of the nourishment composition and the nourishment desideration using an edible machine-learning model.

20. The method of claim 11, wherein generating the nourishment program further comprises:
receiving an intended outcome; and
generating the nourishment program as a function of the intended outcome using a nourishment machine-learning model.
